(19) United States
(12) Patent Application Publication
(10) Pub. No.: US 2007/0173698 A1
(43) Pub. Date: Jul. 26, 2007

(54) Fail-safe risk management system and methods

(76) Inventors: Paul Kivela, Napa, CA (US); Mihir Dalal, Roseville, CA (US); Terry Borchers, Roseville, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/334,770

(22) Filed: Jan. 17, 2006

Related U.S. Application Data
(60) Provisional application No. 60/709,486, filed on Aug. 19, 2005.

(51) Int. Cl. A61B 5/00

(52) U.S. Cl. 600/300; 128/920

(57) ABSTRACT

A medical diagnostic system and methods that use the complaints or symptoms presented by the patient, along with the physician's top-of-mind diagnoses, to identify in real-time a list of fail-safes that correspond to potential high-risk diagnoses that the patient may be suffering from. The fail-safes are preferably listed in rank order with the most serious on down, and serve as a checklist to remind the emergency physician of possible missed high-risk diagnoses, thereby averting errant diagnoses.

### CROSS-REFERENCE TO RELATED APPLICATIONS

This application claims priority from U.S. provisional application Ser. No. 60/709,486 filed on Aug. 19, 2005, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to risk management systems, and more particularly to a system for generating fail-safes.

2. Description of Related Art

In emergency rooms across the U.S., emergency physicians see patients of every description, whom they have typically never seen before, and for whom they must need to diagnose and treat what can be a mind boggling variety of injuries and illnesses in a short period of time.

Patients generally come to the typical emergency room (ER) with, and are often able to communicate, one or more of a number of chief complaints. A triage nurse usually sees the patient first, determines what the major complaints are, and starts the information gathering process. A physician then meets with the patient, asks a variety of questions, gathers additional pertinent information and, based on his/her knowledge and experience, mentally sorts through a large number of diagnoses and identifies the one or more diagnoses at issue. From there, the physician either treats the condition immediately, or orchestrates a variety of medical and laboratory tests, radiology exams, and seeks the advice of medical specialists to learn more, all for the purpose of isolating and otherwise substantiating a diagnosis upon which the proper treatment can be prescribed and the patient either admitted to the hospital or discharged home.

One problem with the above process is that the human mind, even a highly trained physician's mind, is generally incapable of sorting through all of the hundreds of potential diagnoses to identify all of the diagnoses that should be considered and either proven or discarded in treating the patient and the documentation of same.

The practice of medicine is incredibly complex. The odds of human error are great and the pressure is high not to make any mistakes; mistakes that can literally mean the difference between life and death.

Errors that do occur are in most cases recurrent and predictable. The top causes of errors, medical malpractice claims and losses have not changed significantly in the last fifteen years. In the specialty of Emergency Medicine, 90% of the errors involve a failure to diagnose.

Large numbers of medical errors occur in emergency rooms annually. The cost of medical errors is high in terms of human life, short and long-term disability, and attending emotional distress. Of lesser importance are the unnecessary financial costs to patients, as well as the increasing cost of medical malpractice insurance to emergency physicians, the ER groups for whom they practice, as well as the hospitals within which they practice.

To date, neither the advances in medical technology nor the available loss prevention education or training courses have succeeded in lowering the incidence of medical error, or the resulting medical malpractice claims and costs. In fact, large settlements and jury awards are rising unchecked.

### BRIEF SUMMARY OF THE INVENTION

An aspect of the invention is a method for generating one or more fail-safes in response to patient data. In one embodiment, the method comprises the steps of storing a plurality of fail-safes, each fail-safe corresponding to potential high-risk diagnoses, inputting at least one patient complaint, inputting at least one physician diagnosis of the patient, and generating a list of one or more patient fail-safes, wherein the patient fail-safes correlate to either of the inputted patient complaint and the inputted physician diagnosis.

In one embodiment of the current aspect, the method further includes ranking the patient fail-safes based on the severity of the potential high-risk diagnoses associated with each fail-safe. Preferably, the patient fail-safes are numerically weighted according to the severity of the potential high-risk diagnoses associated with each fail-safe.

In some embodiments, the list of patient fail-safes contains fail-safes correlating to both the inputted patient complaint and the inputted physician diagnosis. In a preferred variation, the patient fail-safes are generated as a checklist for review by a physician, wherein the highest ranking fail-safes are placed highest on the checklist.

Generally, inputting at least one patient complaint comprises selecting from a stored list of possible patient complaints. Correspondingly, inputting at least one physician diagnosis comprises selecting from a stored list of possible physician diagnoses.

Another aspect of the invention is a method for generating one or more fail-safes in response to patient data, including the steps of storing a plurality of fail-safes, each fail-safe correlating to potential high-risk diagnoses, and inputting at least one patient complaint. In one embodiment, the method also includes the steps of generating a list of one or more patient fail-safes correlating to the inputted patient complaint, and displaying the patient fail-safes in order based on the severity of the potential high-risk diagnoses associated with each fail-safe.

In some embodiments, the method also includes inputting a physician diagnosis of the patient, and generating a list of one or more patient fail-safes correlating to the inputted physician diagnosis. Hence the list of patient fail-safes may contain fail-safes correlating to both the inputted patient complaint and the inputted physician diagnosis.

In a preferred embodiment, the patient fail-safes are numerically weighted according to the severity of the potential high-risk diagnoses associated with each fail-safe. The patient fail-safes may also be generated as a checklist for review by a physician, wherein the highest ranking fail-safes are placed highest on the checklist. In an alternative embodiment, the patient fail-safes are output in an order based on the severity of the fail-safe and the frequency of the fail-safe occurrence.

Another aspect of the invention is a computer user interface for entering medical data of a patient. The computer user interface comprises a complaint field for entering a complaint expressed by the patient, a diagnosis field for entering a potential diagnosis from the physician, and a pane for displaying one or more fail-safes corresponding to potential high-risk diagnoses for consideration by the physician. Generally, the one or more fail-safes are generated from either an entry in the complaint field or the diagnosis field. Often, the one or more fail-safes are generated from the complaint field and the diagnosis field.

In some embodiments, the complaint field is configured to be populated from a pre-selected list of patient complaints. Correspondingly, the diagnosis field is configured to be populated from a pre-selected list of potential diagnoses. Additionally, the displayed fail-safes may be generated from a list of fail-safes correlating to both the inputted patient complaint and the inputted physician diagnosis.

Preferably, the fail-safes are displayed according to the severity of the potential high-risk diagnoses associated with each fail-safe. In addition, each fail-safe may have a checkbox for recording that the physician considered the fails-safe in treating the patient.

The user interface may also include a patient data field for entering identification data of a patient and a patient discharge field for displaying discharge instructions for the patient.

Yet another aspect is a medical diagnostic system having a user interface for entering and displaying patient data, and a database comprising lists of patient complaints, potential physician diagnoses and fail-safes. The fail-safes correspond to potential high-risk diagnoses, and each fail-safe correlates to at least one complaint or potential diagnosis, wherein the user interface is configured to display a list of fail-safes upon entry of a patient complaint or potential diagnosis.

Preferably, the list of fail-safes are weighted according to severity of the potential high-risk diagnosis associated with each fail-safe, and are displayed in order of ranking with the highest-weighted fail-safes listed first.

The user interface generally comprises a plurality of fields for entering at least one patient complaint and at least one potential physician diagnosis. The plurality of fields are preferably configured to populate from either the list of patient complaints or the list of potential physician diagnoses.

In one embodiment of the current aspect, the database may also comprising a list of discharge instructions, or a list of physicians.

The system may also include a plurality of terminals for displaying the user interface, and a site server configured to store said fail-safe database, patient complaint database, and potential physician diagnoses database. A master server may be coupled to said site server via the Internet. Preferably, the master server is capable of updating the site server.

In yet another aspect, an apparatus is disclosed for generating one or more fail-safes in response to patient data. The apparatus comprises a computer, a database associated with the computer for storing a plurality of fail-safes corresponding to potential high-risk diagnoses. The apparatus also includes means for receiving input of at least one patient complaint, means for receiving input of at least one physician diagnosis of the patient, and means for generating a list of one or more patient fail-safes, wherein the patient fail-safes correlate to either of the inputted patient complaint or the inputted physician diagnosis.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a view of an embodiment of computer user interface in accordance with the present invention.

FIG. 2 is a view of the computer user interface of FIG. 1 with a generated list of fail-safes.

FIG. 3 illustrates an exemplary database architecture in accordance with the present invention.

FIG. 4 illustrates a system diagram of an embodiment of the diagnostic system of the present invention.

FIGS. 5A-5B illustrate a flow diagram of an embodiment of a method of generating fail-safes in accordance with the present invention.

### DETAILED DESCRIPTION OF THE INVENTION

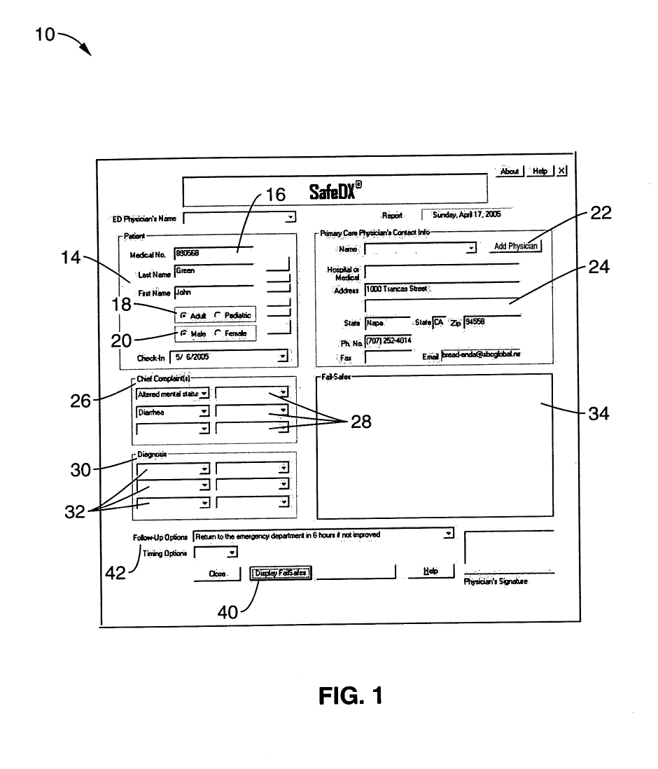

Referring more specifically to the drawings, for illustrative purposes the present invention is embodied in the apparatus generally shown in FIG. 1 through FIG. 5B. It will be appreciated that the apparatus may vary as to configuration and as to details of the parts, and that the method may vary as to the specific steps and sequence, without departing from the basic concepts as disclosed herein.

The system of the present invention, also referred herein as the SafeDX system, uses complaints/symptoms presented by the patient, along with the physician's top-of-mind diagnoses, to identify in real-time a list of fail-safes that correspond to potential high-risk diagnoses that the patient may be suffering from, and preferably list them in rank order with the most serious on down. The list of fail-safes serves as a checklist to remind the emergency physician of possible missed high-risk diagnoses, thereby averting errant diagnoses.

The present invention comprises a medical diagnosis software tool that: 1) gives emergency physicians an effective new way to improve how they practice medicine; 2) standardizes the diagnosis process; 3) prevents/reduces the number and severity of medical errors; 4) produces a consistently defensible medical record; and 5) reduces the number, severity and cost of medical malpractice claims.

At the core of the medical diagnosis system of the present invention are a series of “fail-safes”. Fail-safes, as used and described herein, are checklists, such as those implemented by the airline and nuclear power industries, of mandatory repetitive tasks that when followed avoid the kinds of mistakes or oversights that could lead to catastrophic loss of human life and property.

Although the medical diagnosis system of the present invention may be applied to a number of medical specialties, the SafeDX medical diagnosis system hereinafter described will be illustrated in a configuration directed to the practice of emergency medicine. It is appreciated that the system and methods of the present invention may be similarly applied to other medical needs, such as a general practitioner (e.g. primary care physician), or particular specialties such as internal medicine, neurology, etc.

The fail-safes of the present invention are devised to trigger reminders to physicians based on both the patient's presenting chief complaint and the doctor's potential diagnosis. For example, many patients with inferior myocardial infarctions (heart attacks) may often present with nausea, but not have chest pain. Such patients may often be discharged by the physician with a diagnosis of gastroenteritis (stomach flu). This is a classic mistake, yet one that is recurrent. With the SafeDX system in place, however, the physician is reminded to consider the heart attack diagnosis in all such patients and thereby decrease the incidence of misdiagnosis.

The SafeDX system thus leverages technology to prevent human error and reduce the number and cost of adverse patient outcomes. The system preferably comprises the following components, a graphic (computer) user interface, a database comprising a plurality of tables, computer software and hardware.

Referring now to FIG. 1, the SafeDx graphic user interface 10 in accordance with the present invention is illustrated. The graphic user interface 10 generally comprises a plurality of sections or panels.

First, the interface 10 comprises a patient identification section 12 used to enter patient information, including fields for the patients name 14, medical number 16, adult/pediatric 18, sex 20, etc. The SafeDX system is configured to maintain information on a large number of patients in its database simultaneously.

The user interface may also comprise a physician information section 22 used to enter the contact information of the patient's primary care (or other) physician, with fields 24 for address, phone number and other contact info. As with the patient database, SafeDX is configured to maintain information on a large number of primary care physicians in its database simultaneously.

The user interface 10 also has a chief complaints section 26 comprising a plurality of pull-down fields 28 to enter the patient's primary complaint or complaints. When clicking on any of the pull-down fields 28, a list of possible complaints is displayed, from which the operator may choose from to populate the field.

The user interface 10 also has a physician diagnosis section 30 with a plurality of fields drop down fields 32 to enter the physician's potential diagnosis or diagnoses. As illustrated in FIG. 1, there are six fields for both the chief complaints section 26 and the diagnosis section 30. However, this number may vary depending on the application, and only one field need be entered to generate a list of fail-safes as described below. The pull-down lists of the chief complaints section 26 and the diagnosis section 30 are populated by two separate databases, as described further below.

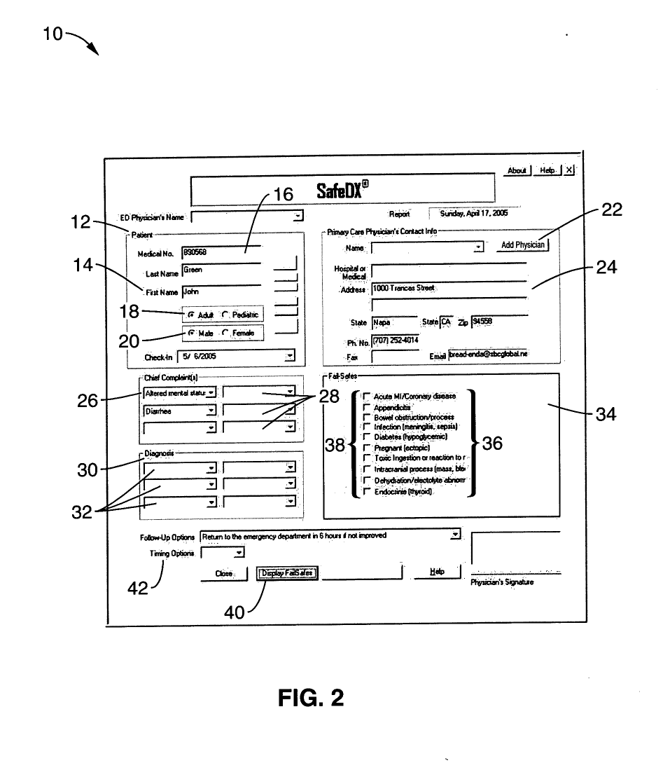

The fail-safes section 34 displays a list of fail-safes generated by the patient's chief complaint(s) and/or the physician's potential diagnosis(es). As shown in FIG. 1, this section is blank until a chief complaint and/or potential diagnosis is entered. Once the operator has entered the desired information in to sections 26 and 30, the “display failsafes” button may be checked, generating a list of fail-safes 36, as illustrated in FIG. 2.

The list of fail-safes 36 are preferably sorted in the order of priority, with the topmost being the most important, i.e., involving the gravest potential medical consequences if missed. Each fail safe also includes a checkbox 38, illustrating that the physician considered each possible diagnosis.

The user interface 10 may also include a follow-up section 42 that may be used to enter the patient's follow-up options and timing in terms of when to return to the emergency department and/or see his/her primary care physician. This section may also be used to select, edit, if necessary, and print a patient-specific set of discharge instructions.

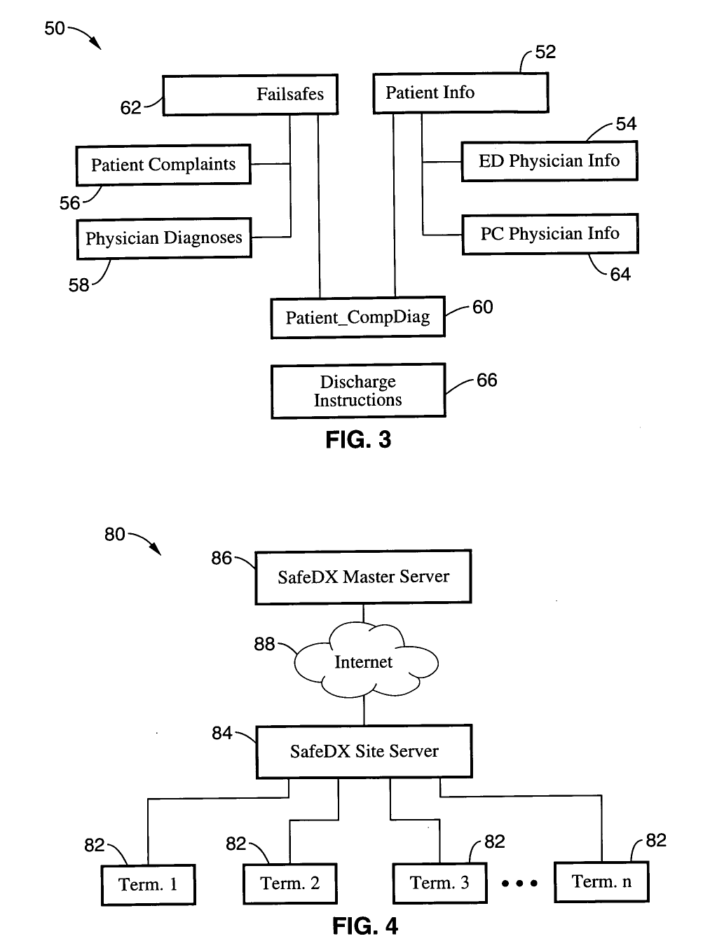

FIG. 3 illustrates the SafeDX system database architecture 50. The database 50 preferably comprises a plurality of tables, each containing data that may be used for the individual modules of the user interface 10 shown in FIGS. 1 and 2. For example, the database 50 may contain an emergency department physician list 54, which may be a list of the emergency physicians practicing in a hospital's emergency department (a single site).

In addition, the database 50 may have a patient information list 52 to hold patient information, including name, medical number, whether they are adult/pediatric, whether they are male/female, etc., which may populate or save data entered in module 12 of user interface 10. The SafeDX system is configured to maintain information on a large number of patients in this database simultaneously.

The database 50 further includes a patient's chief complaint list 56 incorporating a master list of possible patient complaints. This list is preferably used to populate the chief complaint fields 28 shown in FIGS. 1 and 2 upon selection by an operator. Table 1 is an exemplary list of patient complaints for an emergency room scenario. In this example, 29 chief complaints are listed. However, complaints may be deleted or other complaints added to this list as desired.

The database 50 further comprises a physician's potential diagnosis list 58, which incorporates a master list of potential diagnoses. The potential diagnoses list 58 pulls up upon clicking on one of the diagnosis fields 30 of the user interface 10. Table 2 illustrates an exemplary list of potential diagnoses that the physician may select when receiving the patient. Almost 500 potential diagnoses are included in the list shown in Table 2. However, similar to the complaints table above, the number of potential diagnoses can be increased or decreased as necessary.

The patient medical history list 60 is a function of the patient's chief complaints and the physician's potential medical diagnosis history. This table accumulates a historical archive of patient complaint(s) and diagnosis(es) for review by the receiving physician or other physicians.

Finally, the database 50 includes a fail-safe list 62 that incorporates a master list of fail safes. Table 3 is an exemplary list of 38 high-risk diagnoses, all weighted with regard to potential death, short/long term disability or medical malpractice claims and losses. The 38 fail-safes are weighted and ranked numerically with values ranging from 1 to 170. The highest risk diagnosis is acute MI/coronary disease, with a weighted ranking of 1. The lowest risk diagnosis on the fail-safe list is cancer with a weighted rank of 170. Just as with the complaints and diagnoses tables above, the number can be increased or decreased as advisable, as well as the applied weights and rankings.

As can be seen in the exemplary Tables 1 and 2, the each of the chief complaint and physician diagnosis entries have an associated group of one or more fail-safes that correspond to the diagnosis or complaint. There may be as little as one fail-safe, or a large number of fail-safes, associated with a particular complaint (e.g. ear complaints have one fail-safe, where as nausea/vomiting may have a number of fail-safes (5+) generated as a result of its selection). Note should also be taken that the lists shown in Tables 1-3 are directed toward emergency care. For example, a list directed toward the ENT specialty, would have a number of more specific complaints for the general complaint of “ear complaints,” and may not include ER related complaints (e.g. pregnancy) or diagnoses shown in Tables 1 and 2.

The system may also include additional data, including a primary care physician's list 64 to hold the contact information on the primary care physicians in the hospital's trade area. The system can maintain information on a large number of primary care physicians in its database simultaneously.

A discharge instruction list 66 may also be included in database 50 for populating section 42 of the user interface 10. Instruction list 66 preferably comprises a plurality of detailed discharge instructions, each of which may be edited, printed and archived for each and every patient.

The user interface 10 may be configured to operate on a number of different platforms, (e.g., the Microsoft Windows Operating System). The database 50 shown in FIG. 3 is configured to hold/maintain the data that populate the user interface as explained above. Open DataBase Connectivity (ODBC) may be used to connect the lists of database 50 to the master database on the server, explained in more detail below.

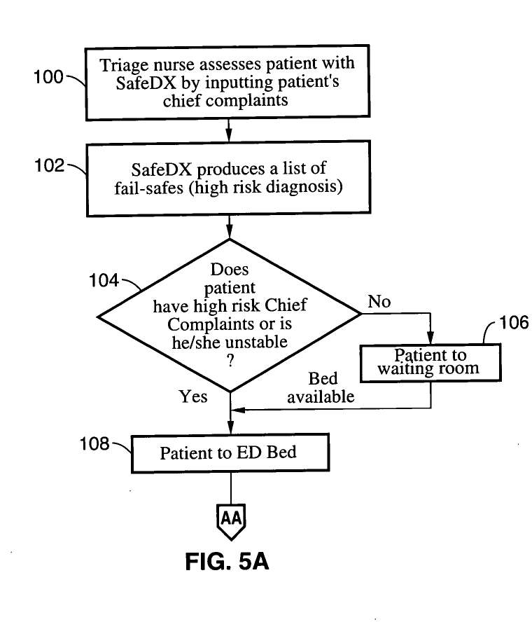

FIG. 4 illustrates an exemplary system configuration 80. The SafeDX system is preferably configured to operate on a number of terminals 82, e.g. a Tablet PC, one for each doctor at a Hospital ED site. The site may be supported wirelessly (or hard-wired) by a SafeDX site server 84. The site server 84 stores all information about the patients, their complaint(s), potential diagnosis(es) and fail-safes. The local site server 84 is preferably connected to a SafeDX master server 86 through an internet connection 88. The SafeDX master server 88 is configured to periodically update all site servers 84 with the latest list of fail-safes and their respective ranks.

The SafeDX system 80 may also be configured to allow periodic update the lists of patient's chief complaints, physician's potential diagnoses and fail-safes, in addition to other database information, remotely over the Internet 88 from a central location such as the master server 86. Patient records are then securely stored and may be quickly and easily retrieved. The SafeDX system 80 is also configured to dovetail with popular patient management software, to allow for seamless integration of computer aided medical services. Daily, weekly, monthly or annual summaries of the patient may be readily accessed and printed upon request.

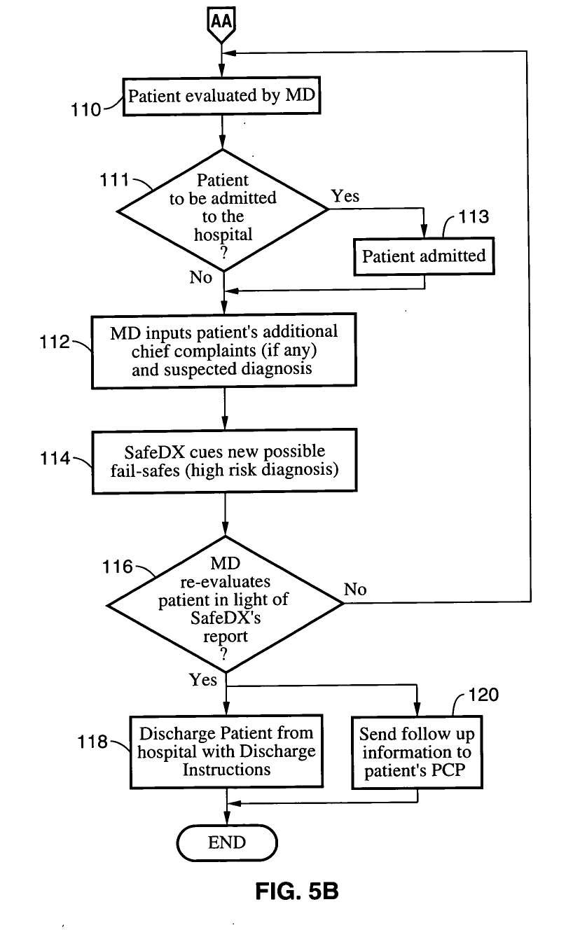

A flow chart of the patient care process and the points in such process at which the SafeDX system is employed is set forth in FIG. 5 as an exemplary method for using the fail-safe diagnostic system of the present invention. The system is configured such that both triage nurses and emergency physicians may access SafeDX at different points in the patient care process.

After arrival at the hospital, a patient will typically see a triage nurse first. Shown as step 100, the triage nurse may then input one or more of the following entries using the tablet PC 82 and SafeDX computer user interface 10:
The patient's identifying data . . . name, medical number, adult/pediatric, male/female.
The patient's chief complaint or complaints.

Because triage nurses cannot diagnose, they will generally not enter any diagnoses. The emergency physician will generally pull up the SafeDX system later in the patient care process, typically after the initial medical evaluation, to make sure he/she hasn't missed a high risk diagnosis(es) and document any diagnoses in the patient's chart.

At step 102, the SafeDX software program takes the patient's chief complaint or complaints entered by the triage nurse, and, in real-time, applies the master list of fail-safes (as shown in Table 3) to each of the chief complaints (Table 1), producing a list of fail-safes applicable to each chief complaint. For example, a chief complaint of “allergic reaction” produces the following three chief complaint fail-safes:

| | | |
|---|---|---|
| Fail-Safe 1 | Toxic ingestion/reaction to medication | 55 |
| Fail-Safe 2 | Airway obstruction (epiglottitis, FB) | 105 |
| Fail-Safe 3 | Brochaspasm/hypoxia | 110 |

Correspondingly, a chief complaint of “weakness” produces the following five chief complaint fail-safes:

| | | |
|---|---|---|
| Fail-Safe 1 | Acute MI/Coronary Disease | 1 |
| Fail-Safe 2 | Appendicitis | 5 |
| Fail-Safe 3 | Pneumothorax | 10 |
| Fail-Safe 4 | Aortic Dissection | 15 |
| Fail-Safe 5 | Diabetes (hypoglycemic) | 45 |

When a patient has more than one chief complaint such as the two above, “allergic reaction” and “weakness”, the SafeDX software program generates a combined list of “fail-safes”, intelligently sorting them in the most appropriate order of priority.

The generated fail-safes may then be used to determine whether the patient is stable or unstable, shown at step 104. If the patient is stable, they are directed to the waiting room (106) to wait for an emergency department (ED) bed, if not, they are admitted to an ED bed (108).

At step 110, the patient is evaluated by the emergency room physician, who then may decide to admit the patient to the hospital (111,113). The physician may then pull up the patient's SafeDX history and input additional chief complaints or one or more potential diagnoses at step 112.

At step 114, the SafeDX software program, again in real-time, applies the master list of fail-safes to each of the potential diagnoses (see Table 2), producing a list of fail-safes applicable to each diagnosis. For example, a suspected diagnosis of “Abnormal EKG” produces the following three potential diagnosis fail-safes:

| | | |
|---|---|---|
| Fail-Safe 1 | Acute MI/Coronary disease | 1 |
| Fail-Safe 2 | Pulmonary Embolus | 12 |
| Fail-Safe 3 | Dysrythymia | 160 |

A suspected diagnosis of “Headache” produces the following five potential diagnosis fail-safes:

| | | |
|---|---|---|
| Fail-Safe 1 | Intracranial process (mass, bleed, trauma) | 60 |
| Fail-Safe 2 | Infection (meningitis, sepsis) | 25 |
| Fail-Safe 3 | CVA/stroke | 75 |
| Fail-Safe 4 | Carbon Monoxide | 150 |
| Fail-Safe 5 | Rhabdomyolosis | 165 |

At step 114, the SafeDX software program, again in real-time, evaluates and combines both the chief complaint fail-safes and the potential diagnoses fail-safes, producing a single list of fail-safes in rank order starting with the most serious first. At step 116, the physician then reevaluates the patient's condition based on the new report. The generated fail-safes in step 114 are all of the diagnoses, given the patient's chief complaint(s) and the physician's suspected diagnosis(es), that the emergency physician will consider in his/her testing, evaluation, and treatment of the patient's condition, and his/her documentation of same.

At step 118, the doctor may then select appropriate patient discharge instructions, follow-up options and timing from SafeDX's database via the discharge section 42 of user interface 10. The physician may also edit the instructions and then print a comprehensive, patient-specific discharge report.

At step 120, the emergency department can further alert the patient's primary care physician, through an automated email, that his/her patient visited the emergency department, the reason for the visit, plus the discharge instructions inclusive of follow-up directions.

In addition to tailoring the SafeDX software/hardware solution to other medical specialties, it is appreciated that the architecture as described above may be implemented in a number of non-medical professions where errors are recurrent, predictable, and preventable. Examples include: accountants, architects, attorneys, contractors, engineers, insurance agents and brokers, realtors, etc.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
Patient's Chief Complaints

| | | | | | | | |
|---|---|---|---|---|---|---|---|
| Complaints | FailSafe_1 | FailSafe_2 | FailSafe_3 | FailSafe_4 | FailSafe_5 | . . . | FailSafe_n |

| | | | | | |
|---|---|---|---|---|---|
| Abdominal Pain | 1 | 5 | 20 | 40 | 50 |
| Allergic reaction | 55 | 105 | 110 | | |
| Altered mental status | 1 | 25 | 45 | 50 | 55 |
| Back pain | 1 | 5 | 20 | 25 | 35 |
| Chest pain | 1 | 3 | 15 | 17 | 10 |
| Diarrhea | 5 | 20 | 77 | | |
| Dizziness/Vertigo | 1 | 25 | 60 | 65 | 75 |
| Dysuria/urinary | 1 | 5 | 20 | 40 | 50 |
| Ear complaints | 45 | | | | |
| EXT trauma | 35 | 80 | 85 | 90 | 95 |
| Eye complaints | 45 | 90 | 95 | 100 | 135 |
| Face & head trauma | 35 | 60 | 90 | 100 | |
| Fever | 5 | 25 | | | |
| Headache (nontraumatic) | 25 | 60 | 65 | 75 | 135 |
| Laceration | 35 | 80 | 85 | 90 | 95 |
| MVA | 35 | 50 | 60 | 80 | 85 |
| Nausea/Vomiting | 1 | 5 | 20 | 25 | 50 |
| Nose bleed | 1 | 115 | 120 | | |
| Pregnancy related | 5 | 50 | 51 | 95 | 120 |
| Psych patients | 1 | 25 | 45 | 50 | 55 |
| Rash (GI bleeding) | 25 | 55 | 110 | | |
| Seizures | 1 | 25 | 45 | 55 | 60 |
| Shortness of breath/ | 1 | 10 | 30 | 105 | 110 |
| Wheezing/Upper | | | | | |
| respiratory | | | | | |
| Soft tissue | 35 | 80 | 85 | 90 | 95 |
| infection/abscess | | | | | |
| Sore throat | 1 | 10 | 30 | 105 | 110 |
| Substance abuse/ | 1 | 25 | 45 | 50 | 55 |
| intoxication/withdrawal | | | | | |
| Syncope | 1 | 25 | 45 | 50 | 55 |
| Vaginal | 5 | 50 | 51 | 95 | 120 |
| bleeding/discharge | | | | | |
| Weakness | 1 | 5 | 10 | 15 | 44 |

TABLE 2
Physician's Potential Diagnoses

| | | | | | | | |
|---|---|---|---|---|---|---|---|
| Diagnosis | FailSafe_1 | FailSafe_2 | FailSafe_3 | FailSafe_4 | FailSafe_5 | . . . | FailSafe_n |

| | | | | | |
|---|---|---|---|---|---|
| AAA | 1 | 5 | 15 | | |
| Abdominal mass | 1 | 5 | 20 | 40 | 50 |
| Abdominal Pain | 1 | 5 | 20 | 40 | 50 |
| Abnl. Lung finding | 1 | 10 | 12 | 30 | 170 |
| Abnormal blood | 77 | 160 | 30 | | |
| Chemistry | | | | | |
| Abnormal EKG | 1 | 12 | 160 | | |
| Abortion, missed | 50 | 30 | 5 | | |
| Abortion, sponateous | 50 | 30 | 5 | | |
| Abortion, threatened | 50 | 30 | 5 | | |
| Abrasion | 35 | 85 | 80 | 90 | 95 |
| Abrasion, finger | 35 | 85 | 80 | 90 | 95 |
| Abrasion, foot/toe | 35 | 85 | 80 | 90 | 95 |
| Abrasion, forearm | 35 | 85 | 80 | 90 | 95 |
| Abrasion, hand | 35 | 85 | 80 | 90 | 95 |
| Abrasion, head | 60 | 25 | 100 | 95 | |
| Abrasion, leg/hip | 35 | 85 | 80 | 90 | 95 |
| Abrasion, shoulder/arm | 35 | 85 | 80 | 90 | 95 |
| Abrasion, trunk | 10 | 95 | 80 | | |
| Abrasions | 35 | 85 | 80 | 90 | 95 |
| Abscess | 30 | 35 | 85 | 80 | 90 |
| Abscess, peritonsillar | 105 | 30 | | | |
| Abscess, rectal/anus | 30 | 95 | 20 | | |
| Adverse Effect | 110 | 105 | 55 | | |
| Medication | | | | | |
| Alcohol Abuse | 77 | 45 | 100 | | |
| Alcohol Intoxication | 77 | 145 | 100 | 115 | 60 |
| Allergy | 110 | 105 | 55 | | |
| Altered Mental Status | 1 | 45 | 55 | 60 | 25 |
| Amputation, finger | 35 | 85 | 95 | 80 | |
| Amputation, thumb | 35 | 85 | 95 | 80 | |
| Anaphylactic shock | 1 | 55 | 105 | 110 | |
| Anaphylaxis to sting | 110 | 105 | 55 | | |
| Anemia | 115 | 120 | 1 | | |
| Angina | 1 | 10 | 12 | 15 | 85 |
| Angioedema | 110 | 105 | 55 | 30 | |
| Anxiety | 1 | 30 | 45 | 50 | 55 |
| Anxiety/panic attack | 1 | 30 | 45 | 50 | 55 |
| Aortic dissection | 1 | | | | |
| Aphasia | 60 | 25 | 75 | 150 | 165 |
| Aphthous oral ulcer | 30 | | | | |
| Appendicitis | 30 | | | | |
| Appetite, Lack/Loss | 170 | 60 | 25 | 75 | 77 |
| Arthritis | 80 | 85 | 100 | 35 | |
| Ascites | 77 | 115 | 120 | 30 | 45 |
| Aspiration | 75 | 30 | 105 | 110 | 1 |
| Assault | | | | | |
| Asthma | 1 | 12 | 110 | 30 | 105 |
| Asthma with status | 1 | 12 | 110 | 30 | 105 |
| Atrial Fibrillation | 1 | 55 | 160 | | |
| Atrial Flutter | 1 | 55 | 160 | | |
| AV block | 1 | 55 | 160 | | |
| Back Ache | 125 | 40 | 20 | 1 | 5 |
| Back Pain, Low | 125 | 40 | 20 | 1 | 5 |
| Bartholin's Abscess | 30 | | | | |
| Bell's Palsy | 55 | 60 | 25 | | |
| Biliary Disease | 1 | 5 | 20 | 50 | 40 |
| Bipolar Disease | 55 | 145 | 45 | 30 | 77 |
| Bite, animal | 95 | 90 | 35 | 85 | 80 |
| Bite, dog | 95 | 90 | 35 | 85 | 80 |
| Blood/Body fluid | 30 | | | | |
| exposure | | | | | |
| Blurred Vision | 45 | 55 | 60 | 25 | 100 |
| Bowel Obstruction | 40 | 170 | 5 | 30 | |
| Brain injury, closed | 100 | | | | |
| Brain injury, open | 100 | 95 | | | |
| Broken tooth | 90 | 100 | 60 | 35 | 105 |
| Bronchiolitis | 110 | 30 | 105 | | |
| Bronchitis | 110 | 1 | 12 | 30 | 105 |
| Bronchospasm | 110 | 1 | 12 | 30 | 105 |
| Burn | 95 | 105 | 150 | 115 | 100 |
| Burn, BSA 10-19% | 95 | 150 | 105 | 115 | 100 |
| Burn, BSA 20-29% | 95 | 150 | 105 | 115 | 100 |
| Burn, BSA 30-39% | 95 | 150 | 105 | 115 | 100 |
| Burn, BSA 40-49% | 95 | 150 | 105 | 115 | 100 |
| Burn, BSA 50-59% | 95 | 150 | 105 | 115 | 100 |
| Burn, BSA 60-69% | 95 | 150 | 105 | 115 | 100 |
| Burn, BSA 70-79% | 95 | 150 | 105 | 115 | 100 |
| Burn, BSA 80-89% | 95 | 150 | 105 | 115 | 100 |
| Burn, BSA > 90% | 95 | 150 | 105 | 115 | 100 |
| Burn, BSA < 10% | 95 | 150 | 105 | 115 | 100 |
| Bursitis | 35 | 80 | 85 | 100 | |
| Bursitis, Knee | 35 | 80 | 85 | 100 | |
| Bursitis, Olecranon | 35 | 80 | 85 | 100 | |
| Bursitis, Shoulder | 35 | 80 | 85 | 100 | |
| Bursitis, Wrist | 35 | 80 | 85 | 100 | |
| Carbon Monoxide | 145 | | | | |
| Inhalation | | | | | |
| Cardiac Arrest | | | | | |
| Cast Removal | 100 | | | | |
| Cellulitis | 35 | 100 | 12 | 30 | 95 |
| Cellulitis, arm | 35 | 100 | 12 | 30 | 95 |
| Cellulitis, buttocks | 35 | 100 | 12 | 30 | 95 |
| Cellulitis, facial | 35 | 100 | 12 | 30 | 95 |
| Cellulitis, finger | 35 | 100 | 12 | 30 | 95 |
| Cellulitis, foot | 35 | 100 | 12 | 30 | 95 |
| Cellulitis, hand | 35 | 100 | 12 | 30 | 95 |
| Cellulitis, leg | 35 | 100 | 12 | 30 | 95 |
| Cellulitis, neck | 35 | 100 | 12 | 30 | 95 |
| Cellulitis, toe | 35 | 100 | 12 | 30 | 95 |
| Cellulitis, trunk | 35 | 100 | 12 | 30 | 95 |
| Cerumen impaction | 30 | | | | |
| Chalazion | 100 | | | | |
| Chest pain | 1 | 10 | 12 | 15 | 85 |
| Chest pain, pleuritic | 1 | 10 | 12 | 15 | 85 |
| Chest wall pain | 1 | 10 | 12 | 15 | 85 |
| CHF | 1 | 12 | 15 | 30 | 110 |
| Chicken pox | 30 | 110 | 95 | | |
| Cholecystitis | 5 | 50 | | | |
| Cirrhosis | 30 | 115 | 120 | 20 | |
| Cocaine abuse | 45 | 55 | 145 | 1 | |
| Concussion | 100 | 60 | | | |
| Conjunctivitis | 100 | 30 | | | |
| Constipation | 40 | 20 | 1 | | |
| Contusion | 35 | 80 | 90 | 85 | |
| Contusion, abdomen | 100 | | | | |
| Contusion, arm | 35 | 80 | 85 | 90 | |
| Contusion, back | | | | | |
| Contusion, chest wall | 35 | 100 | 80 | 10 | 1 |
| Contusion, eye | 100 | 95 | 90 | | |
| Contusion, face/scal/neck | 60 | 100 | 95 | 90 | 35 |
| Contusion, finger | 35 | 80 | 85 | 90 | |
| Contusion, foot | 35 | 80 | 85 | 90 | |
| Contusion, hand | 35 | 80 | 85 | 90 | |
| Contusion, hip | 35 | 80 | 85 | 90 | |
| Contusion, knee | 35 | 80 | 85 | 90 | |
| Contusion, leg lower | 35 | 80 | 85 | 90 | |
| Contusion, shoulder | 35 | 80 | 85 | 90 | |
| Contusion, thigh | 35 | 80 | 85 | 90 | |
| COPD | 110 | 1 | 30 | 10 | 105 |
| Corneal abrasion | 90 | 165 | 100 | 95 | |
| costochondritis | 1 | 10 | 12 | 15 | 85 |
| Cough | 110 | 30 | 1 | 10 | 105 |
| Croup | 110 | 30 | 1 | 10 | 105 |
| Crush inj., arm | 35 | 18 | 80 | 85 | 90 |
| Crush inj., back | 125 | 35 | 100 | | |
| Crush inj., buttock | 125 | 100 | 35 | 20 | |
| Crush inj., genitalia | 100 | 35 | 80 | | |
| Crush inj., leg | 35 | 18 | 80 | 85 | 90 |
| Crush inj., neck | 35 | 100 | 105 | | |
| Crush inj., scalp/face | 60 | 35 | 105 | | |
| Crush inj., trunk | 10 | 110 | 1 | 35 | 100 |
| CVA | 115 | 120 | 1 | 45 | 55 |
| Cyanosis | 105 | 110 | 1 | 10 | 25 |
| Dehydration/Hypovolemia | 25 | 1 | 5 | 20 | 77 |
| Dental abscess | 105 | 30 | 95 | 100 | |
| Dental caries | 30 | 105 | 95 | 100 | |
| Dental disorder | 30 | 105 | 95 | 100 | |
| Depression | 45 | 55 | 60 | 25 | 130 |
| Dermatitis | 55 | 25 | 110 | | |
| Diabetes Type 1 | 95 | 45 | 25 | 1 | |
| Diabetes Type 2 | 95 | 45 | 25 | 1 | |
| Diarrhea | 5 | 20 | 77 | | |
| Disloc., ankle closed | 35 | 85 | 80 | 90 | |
| Disloc., elbow closed | 35 | 85 | 80 | 90 | |
| Disloc., finger closed | 35 | 85 | 80 | 90 | |
| Disloc., finger open | 35 | 85 | 80 | 90 | |
| Disloc., foot closed | 35 | 85 | 80 | 90 | |
| Disloc., hip closed | 35 | 85 | 80 | 90 | |
| Disloc., jaw closed | 100 | 60 | 105 | 35 | |
| Disloc., knee closed | 35 | 85 | 80 | 90 | |
| Disloc., patella closed | 35 | 85 | 80 | 90 | |
| Disloc., shoulder closed | 35 | 85 | 80 | 90 | |
| Disloc., wrist closed | 35 | 85 | 80 | 90 | |
| Diverticulitis | 40 | 20 | 1 | 5 | 50 |
| dizziness | 45 | 55 | 60 | 25 | 130 |
| Drug abuse | 45 | 55 | 60 | 25 | 130 |
| DVT | 80 | 12 | | | |
| Dysarthia | 45 | 55 | 60 | 25 | 130 |
| Dysmennorhea | 50 | 120 | 5 | 95 | |
| Dyspareunia | 50 | 25 | 5 | 95 | 140 |
| Dysphagia | 1 | 10 | 12 | 15 | 85 |
| Dysthmia | 55 | 60 | 25 | 130 | 1 |
| Dysuria | 40 | 20 | 1 | 5 | 50 |
| Ectopic pregnancy | 120 | 5 | | | |
| Eczema | 95 | 55 | | | |
| Edema | 1 | 12 | 77 | 110 | |
| Electrocution | 1 | 77 | | | |
| Electrolyte imbalance | 77 | 1 | 25 | 30 | 60 |
| Emphysema | 110 | 30 | 1 | 10 | 12 |
| Endocarditis | 1 | 30 | 35 | | |
| Epididymitis | 30 | 70 | | | |
| Epistaxis | 115 | 1 | 120 | | |
| Esophageal reflux | 1 | 10 | 12 | 15 | 85 |
| Esophagitis | 1 | 10 | 12 | 15 | 85 |
| Exam post condition | | | | | |
| Eye disorder | 165 | 45 | 90 | 100 | 95 |
| Falon | 95 | 35 | 80 | 90 | 100 |
| Fatigue/malaise | 45 | 55 | 60 | 25 | 130 |
| Febrile seizure | 25 | 5 | 60 | | |
| Fecal impaction | 20 | 5 | 77 | | |
| Fever | 25 | 30 | 5 | | |
| Foley replacement | 77 | 35 | | | |
| Follow-up exam | | | | | |
| Foreign body, conjunctiva | 165 | 90 | 100 | 95 | |
| Foreign body, cornea | 165 | 90 | 100 | 95 | |
| Foreign body, eye | 165 | 90 | 100 | 95 | |
| Foreign body, finger | 35 | 85 | 80 | 90 | 18 |
| Foreign body, foot/toe | 35 | 85 | 80 | 90 | 18 |
| Foreign body, forearm | 35 | 85 | 80 | 90 | 18 |
| Foreign body, hand | 35 | 85 | 80 | 90 | 18 |
| Foreign body, leg/hip | 35 | 85 | 80 | 90 | 18 |
| Foreign body, mouth | 110 | 90 | | | |
| Foreign body, should/arm | 35 | 85 | 80 | 90 | 18 |
| Foreign body, superficial | 35 | 85 | 80 | 90 | 18 |
| Foreign body, truck | 10 | 95 | 110 | | |
| Foreign body, bronchus | 110 | 90 | 105 | | |
| Foreign body, ear | 90 | 95 | | | |
| Foreign body, esophagus | 110 | 105 | 90 | | |
| Foreign body, nose | 105 | 110 | 90 | | |
| Foreign body, rectum | 90 | 95 | | | |
| Foreign body, stomach | 90 | 20 | | | |
| Foreign body, trachea | 90 | 105 | 110 | 95 | |
| Foreign body, vagina | 30 | 35 | 90 | 95 | 140 |
| Fx closed | 35 | 85 | 80 | 90 | 18 |
| Fx, ankle closed | 35 | 85 | 80 | 90 | 18 |
| Fx, arm closed | 35 | 85 | 80 | 90 | 18 |
| Fx, cervical spine closed | 125 | 35 | 100 | | |
| Fx, clavicle closed | 10 | 80 | 100 | | |
| Fx, colles closed | 35 | 85 | 80 | 90 | 95 |
| Fx, face closed | 110 | 105 | 60 | 35 | |
| Fx, femur closed | 35 | 85 | 80 | 90 | 18 |
| Fx, fibula closed | 35 | 85 | 80 | 90 | 18 |
| Fx, fibula open | 35 | 85 | 80 | 90 | 18 |
| Fx, finger closed | 35 | 85 | 80 | 90 | 18 |
| Fx, finger open | 35 | 85 | 80 | 90 | 18 |
| Fx, foot closed | 35 | 85 | 80 | 90 | 18 |
| Fx, foot open | 35 | 85 | 80 | 90 | 18 |
| Fx, forearm closed | 35 | 85 | 80 | 90 | 18 |
| Fx, humerus closed | 35 | 85 | 80 | 90 | 18 |
| Fx, lumbar spine closed | 125 | 35 | 100 | 95 | |
| Fx, mandible closed | 110 | 100 | 35 | 105. | 60 |
| Fx, metacarpal closed | 35 | 85 | 80 | 90. | 18 |
| Fx, metacarpal open | 35 | 85 | 80 | 90 | 18 |
| Fx, nose closed | 60 | 35 | 100 | 80 | |
| Fx, nose open | 60 | 35 | 100 | 80 | |
| Fx, patella closed | 35 | 85 | 80 | 90 | 18 |
| Fx, pelvis closed | 35 | 100 | 80 | | |
| Fx, scapula closed | 10 | 80 | 100 | 35 | |
| Fx, skull closed | 60 | 35 | 100 | | |
| Fx, skull open | 60 | 35 | 100 | 95 | |
| Fx, thoracic spine closed | 125 | 35 | 100 | 95 | |
| Fx, tibia closed | 35 | 85 | 80 | 90 | 18 |
| Fx, tibia open | 35 | 85 | 80 | 90 | 18 |
| Fx, tibia/fibula closed | 35 | 85 | 80 | 90 | 18 |
| Fx, tibia/fibula open | 35 | 85 | 80 | 90 | 18 |
| Fx, wrist carpal closed | 35 | 85 | 80 | 90 | 18 |
| Gait abnormality | 75 | 45 | 55 | 60 | 25 |
| Gangrene | 80 | 95 | 30 | 35 | 45 |
| Gastritis | 40 | 20 | 1 | 5 | 50 |
| Gastroenteritis | 40 | 20 | 1 | 5 | 50 |
| GI bleeding | 40 | 115 | 120 | 20 | |
| Gingivitis | 30 | 95 | | | |
| Gonorrhea, lower GU | 50 | 140 | | | |
| Gout | 30 | 35 | | | |
| G-tube replacement | 20 | 35 | | | |
| Hallucinations | 45 | 55 | 60 | 25 | 145 |
| Headache | 60 | 25 | 75 | 150 | 165 |
| Headache, migraine | 60 | 25 | 75 | 150 | 165 |
| Healthy child exam | | | | | |
| Heart dis, not specified | 1 | 10 | 12 | 15 | 85 |
| Heartburn | 1 | 10 | 12 | 15 | 85 |
| Heat exhaustion | 77 | 1 | | | |
| Heat stroke | 77 | 1 | | | |
| Hematemesis | 20 | 1 | 115 | 120 | |
| Hematuria | 30 | 115 | 120 | 100 | |
| Heme + stool | 20 | 115 | 120 | | |
| Hemiparesis | | | | | |
| Hemoptysis | 115 | 30 | 110 | 12 | 120 |
| Hemorrhoids | 20 | | | | |
| Hemothorax, closed | 120 | 110 | 105 | 100 | 10 |
| trauma | | | | | |
| Hemothorax, open | 120 | 110 | 105 | 100 | 10 |
| trauma | | | | | |
| Hernia | 20 | | | | |
| Herpes simplex | 30 | 140 | | | |
| Herpes zoster | 30 | 95 | | | |
| Herpes, vulvovag | 140 | 30 | 95 | | |
| Hiccough | 60 | 1 | 45 | 60 | 75 |
| Hyperglycemia | 45 | 1 | | | |
| Hypertension | 1 | 15 | | | |
| Hyperventilation | 1 | 10 | 12 | 60 | |
| Hyphema | 35 | 100 | 80 | | |
| Hypoglycemia | 45 | | | | |
| Hypotension | 1 | 30 | 35 | 120 | 40 |
| Hypoxia | 105 | 110 | 1 | 30 | 35 |
| Impetigo | 55 | 25 | 95 | | |
| Inguinal hernia | 20 | | | | |
| Injury | | | | | |
| Injury, abdomen internal | 20 | 35 | 100 | | |
| Injury, face/neck | 90 | 100 | 60 | 35 | |
| Injury, finger | 35 | 80 | 85 | 90 | 18 |
| Injury, forearm | 35 | 80 | 85 | 90 | 18 |
| Injury, hand | 35 | 80 | 85 | 90 | 18 |
| Injury, head | 60 | 100 | 35 | 105 | |
| Injury, hip/thigh | 100 | 35 | 120 | | |
| Injury, leg lower | 35 | 80 | 85 | 90 | 18 |
| Injury, multiple sites | 100 | 35 | 120 | | |
| Injury, shoulder/arm | 35 | 80 | 85 | 90 | 18 |
| Injury, thoracic | 10 | 100 | 35 | | |
| Injury, trunk | 10 | 100 | 35 | | |
| Insect bite | 90 | 18 | | | |
| Intracerebral hemorr. | 60 | 100 | 35 | 115 | 105 |
| Intracranial contusion | 60 | 100 | 35 | 115 | 105 |
| Intracranial hemorr traum | 60 | 100 | 35 | 115 | 105 |
| Intra-cranial hemorrhage | 60 | 100 | 35 | 115 | 105 |
| Intussusception | 20 | 30 | 77 | | |
| Iritis | 165 | 45 | 90 | 100 | 95 |
| Jaundice | 115 | 120 | 20 | | |
| Keratitis | 165 | 45 | 90 | 100 | 95 |
| Knee, internal | 35 | 85 | 80 | 95 | |
| derangemen | | | | | |
| Laryngitis | 105 | 30 | 110 | | |
| Lice | | | | | |
| Lupus | 115 | 120 | 77 | 30 | |
| Lymphadenitis | 95 | 30 | 25 | | |
| Lymphangitis | 95 | 30 | 25 | | |
| Medical exam | | | | | |
| Medication refill | 45 | 55 | 145 | | |
| Melena | 20 | 115 | 120 | 77 | |
| Meningitis | 25 | 30 | 95 | 60 | |
| Mentstrual disorder | 50 | 120 | 5 | 95 | 140 |
| MI | 1 | 10 | 12 | 15 | 85 |
| Migraine Headache | 60 | 25 | 75 | 150 | 165 |
| Muscle spasm | 125 | 35 | 100 | 1 | |
| MVA | 60 | 125 | 100 | 35 | 80 |
| MVA, driver | 60 | 125 | 100 | 35 | 80 |
| MVA, motorcycle | 60 | 125 | 100 | 35 | 80 |
| MVA, motorcycle | 60 | 125 | 100 | 35 | 80 |
| passenger | | | | | |
| MVA, passenger | 60 | 125 | 100 | 35 | 80 |
| MVA, pedestrian | 60 | 125 | 100 | 35 | 80 |
| Myalgia | 35 | 95 | 165 | | |
| Nausea | 50 | 20 | 1 | 77 | 50 |
| Nausea w/vomiting | 50 | 20 | 1 | 77 | 50 |
| Near drowning | 110 | 105 | | | |
| Neck sprain | 125 | 35 | 100 | | |
| Needlestick injury | 95 | | | | |
| Neglect, child | 35 | 100 | 55 | | |
| Nephrolithiasis | 40 | 20 | 5 | 1 | 50 |
| Neuralgia/neuritis | 125 | 35 | 60 | 100 | |
| Neuropathy, peripheral | 125 | 35 | 60 | 100 | |
| New onset seizure | 45 | 55 | 60 | 25 | 130 |
| Observation after acciden | | | | | |
| Oral soft tissue disease | 95 | 110 | 105 | | |
| Orchitis/Epididymitis | 70 | 30 | 95 | | |
| Osteomyelitis | 35 | 95 | 30 | | |
| Otalgia | 45 | 95 | 90 | | |
| Otitis externa | 45 | 95 | 90 | | |
| Otitis media | 45 | 95 | 90 | | |
| Overdose | 45 | 55 | 60 | 130 | 1 |
| Pacemaker malfunction | 1 | | | | |
| Pain, abdominal | 40 | 20 | 1 | 5 | 50 |
| Pain, breast | 95 | 30 | 100 | | |
| Pain, eye | 165 | 90 | 45 | 100 | 95 |
| Pain, face | 60 | 35 | 100 | 30 | |
| Pain, joint | 35 | 85 | 80 | 90 | 95 |
| Pain, limb | 35 | 85 | 80 | 90 | 95 |
| Pain, rectum/anal | | | | | |
| Palpatations | 1 | 77 | 160 | | |
| Pancreatitis, acute | | | | | |
| Paranoia | 45 | 55 | 60 | 130 | 1 |
| Parkinson's disease | | | | | |
| Paronychia, finger | 35 | 80 | 85 | 90 | 95 |
| Paronychia, tos | 35 | 80 | 85 | 90 | 95 |
| Peptic ulcer disease | 1 | 40 | 20 | 5 | 50 |
| Perforated TM | 95 | 60 | | | |
| Pericarditis | 1 | 30 | 10 | 12 | 15 |
| Peritonitis | 115 | 30 | 120 | 77 | 20 |
| Pharyngitis | 105 | 110 | 30 | | |
| Placenta previa | 50 | 115 | 120 | | |
| Pleurisy | 110 | 85 | 1 | 12 | 10 |
| Pneumonia | 110 | 85 | 1 | 12 | 10 |
| Pneumothorax, closed trau | 1 | 110 | 12 | 30 | 100 |
| Pneumothorax, open trauma | 1 | 110 | 12 | 95 | 100 |
| Pnuemothorax, spont. | 1 | 110 | 12 | 30 | 100 |
| Post-concussion | 60 | 55 | | | |
| syndrome | | | | | |
| Preeclampsia | 155 | 77 | 60 | 50 | |
| Pregnancy | 5 | 50 | | | |
| Pregnancy test (neg) | 5 | | | | |
| Priapism | 115 | | | | |
| Pruritus | 45 | 55 | | | |
| Psoriasis | 55 | 25 | 95 | | |
| PSVT | 1 | 77 | 160 | | |
| Psychosis | 45 | 1 | 55 | 60 | 25 |
| Pulmonary contusion | 1 | 10 | 12 | 15 | 85 |
| Pulmonary edema | 1 | 10 | 12 | 15 | 85 |
| Pulmonary embolism | 1 | | | | |
| Pyelonephritis | 5 | 40 | 20 | 25 | 50 |
| Rape | 140 | 100 | 145 | | |
| Rash | 55 | 25 | 110 | | |
| Renal colic | 40 | 20 | 1 | 5 | 50 |
| Renal failure, acute | 45 | 1 | 77 | | |
| Renal failure, chronic | 45 | 1 | 77 | | |
| Renal insufficiency | 45 | 1 | 77 | | |
| Respiratory arrest | 110 | 30 | 1 | 10 | 105 |
| Respiratory distress | 110 | 30 | 1 | 10 | 105 |
| Respiratory failure | 110 | 30 | 1 | 10 | 105 |
| Rhinitis, allergic | 30 | 110 | 105 | 60 | |
| Rupture achilles tendon | 35 | 85 | 80 | 95 | 100 |
| Rupture patellar tendon | 35 | 85 | 80 | 95 | 100 |
| SAH | 120 | 115 | 60 | 100 | |
| Scabies | | | | | |
| Scarlet fever | | | | | |
| Schizophrenia | 45 | 55 | 60 | 25 | 130 |
| Sciatica | 125 | 60 | 40 | 35 | 100 |
| Seizure | 45 | 55 | 60 | 25 | 130 |
| Sensory prob. Head | 60 | 30 | 100 | | |
| Sensory prob. Limbs | 125 | 60 | 80 | 55 | |
| Sensory prob. Neck/trunk | 125 | 60 | 1 | 80 | 55 |
| Sepsis | 1 | 165 | 25 | 30 | |
| Shock | 1 | 120 | 25 | 30 | 100 |
| Shortness of breath | 110 | 30 | 1 | 10 | 105 |
| Sickle cell desease | 95 | 30 | 110 | 120 | |
| Sinusitis | 35 | 30 | 60 | 95 | 105 |
| Skin Disorder | 55 | 25 | 110 | 95 | |
| Sore Throat | 105 | 110 | 30 | | |
| Spinal cord injury | 125 | 100 | | | |
| Stomatitis | 105 | 110 | 30 | | |
| Str/sprain | 35 | 85 | 80 | 90 | 18 |
| Str/sprain, ankle | 35 | 85 | 80 | 90 | 18 |
| Str/sprain, finger/hand | 35 | 85 | 80 | 90 | 18 |
| Str/sprain, foot | 35 | 85 | 80 | 90 | 18 |
| Str/sprain, knee/leg | 35 | 85 | 80 | 90 | 18 |
| Str/sprain, lumbar | 125 | 40 | 20 | 1 | 5 |
| Str/sprain, neck | 125 | 40 | 20 | 1 | 110 |
| Str/sprain, shoulder/arm | 35 | 85 | 80 | 90 | 18 |
| Str/sprain, thoracic | 1 | 10 | 12 | 15 | 85 |
| Str/sprain, wrist | 35 | 85 | 80 | 90 | 18 |
| Strep Throat | 110 | 30 | 1 | 105 | 10 |
| Stridor | 110 | 30 | 10 | 105 | |
| Subdural hemorrhage | 60 | 115 | 120 | 95 | 55 |
| Suicide gesture | 45 | 55 | 60 | 25 | 130 |
| Suicide ideation/attempt | 45 | 55 | 60 | 25 | 130 |
| Sunburn | 95 | 77 | | | |
| Suture removal | | | | | |
| Swelling, limb | 35 | 100 | 12 | 80 | |
| Swelling, throat | 110 | 30 | 105 | 55 | |
| Syncope | 45 | 55 | 60 | 25 | 130 |
| Tachycardia | 1 | 12 | 30 | 160 | |
| Tachypnea | 110 | 30 | 1 | 10 | 12 |
| Tendon lac, foot | 35 | 85 | 80 | 90 | 95 |
| Tendon lac, forearm | 35 | 85 | 80 | 90 | 95 |
| Tendon lac, hand | 35 | 85 | 80 | 90 | 95 |
| Tension headache | 60 | 25 | 75 | 150 | 165 |
| Threatened miscarriage | 50 | 120 | | | |
| Thrombocytopenia | 115 | 120 | 25 | | |
| Thrush | 110 | 30 | 10 | 105 | 95 |
| TIA | 69 | 45 | 75 | 1 | |
| Tinnitus | 60 | 25 | 75 | | |
| TMJ pain | 100 | 1 | | | |
| Torticollis | 125 | 35 | | | |
| Tremor | 60 | 55 | 75 | | |
| Trichomonias | 50 | 120 | 140 | 95 | |
| Urethritis | 140 | 25 | 95 | 5 | 140 |
| URI | 110 | 30 | 1 | 10 | 12 |
| Urinary incontinence | 45 | 55 | 60 | 25 | 130 |
| Urinary retention | 45 | 55 | 60 | 25 | 130 |
| Uriticaria | 55 | 25 | 110 | | |
| UTI | 40 | 5 | 20 | 50 | 140 |
| Vaginal bleeding | 50 | 120 | 5 | 95 | 77 |
| Vaginal discharge | 50 | 140 | 5 | 95 | 77 |
| Vertigo, central | 75 | 25 | 60 | 65 | 100 |
| Vertigo, peripheral | 75 | 25 | 60 | 65 | 100 |
| Viral exanthem | 25 | 55 | 110 | | |
| Viral syndrome | 25 | | | | |
| Visual problem | 165 | 45 | 100 | 95 | 60 |
| Vomiting | 5 | 20 | 1 | 77 | 50 |
| Vomiting of pregnancy | 50 | 5 | 77 | 25 | |
| Weakness, limbs | 45 | 55 | 60 | 25 | 130 |
| Wheezing | 110 | 30 | 1 | 10 | 12 |
| Withdrawal, alcohol | 45 | 55 | 60 | 25 | 1 |
| Withdrawal, drug | 45 | 55 | 60 | 25 | 1 |
| Wound | 35 | 85 | 80 | 90 | 18 |
| Wound (operative) infec. | 35 | 85 | 80 | 90 | 18 |
| Wound (trauma) infection | 35 | 85 | 80 | 90 | 18 |
| Wound check | 35 | 85 | 80 | 90 | 18 |
| Wound, abdomen | 85 | 100 | 90 | 18 | |
| Wound, arm upper | 35 | 85 | 80 | 90 | 18 |
| Wound, back | 125 | 40 | 25 | 90 | 18 |
| Wound, chest wall | 1 | 10 | 35 | 80 | 90 |
| Wound, ear | 45 | 100 | 95 | | |
| Wound, elbow | 35 | 85 | 80 | 90 | 18 |
| Wound, eye globe | 90 | 100 | 45 | 95 | |
| Wound, face | 95 | 100 | 45 | 90 | |
| Wound, finger | 35 | 85 | 80 | 90 | 18 |
| Wound, fingernail | 35 | 85 | 80 | 90 | 18 |
| Wound, foot | 35 | 85 | 80 | 90 | 18 |
| Wound, forearm | 35 | 85 | 80 | 90 | 18 |
| Wound, forehead | 45 | 100 | 95 | 90 | |
| Wound, hand | 35 | 85 | 80 | 90 | 18 |
| Wound, hip/thigh | 35 | 85 | 80 | 90 | 18 |
| Wound, knee/leg/ank | 35 | 85 | 80 | 90 | 18 |
| Wound, mouth | 18 | 90 | 95 | 100 | 105 |
| Wound, neck | 18 | 90 | 95 | 100 | 105 |
| Wound, penis | 35 | 100 | 80 | 18 | |
| Wound, scalp | 45 | 100 | 90 | 95 | |
| Wound, shoulder | 35 | 85 | 80 | 80 | 18 |
| Wound, toe | 35 | 85 | 80 | 90 | 18 |
| Wound, toenail | 35 | 85 | 80 | 90 | 18 |
| Wound, wrist | 35 | 85 | 80 | 90 | 18 |
| Yeast inf., vaginal | 50 | 5 | 95 | 140 | |

TABLE 3
Fail-Safes

| FailSafe | Weighted Rank |
|---|---|
| Acute MI/Coronary disease | 1 |
| Appendicitis | 5 |
| Pneumothorax | 10 |
| Pulmonary Embolus | 12 |
| Aortic Disscetion | 15 |
| Esophageal Rupture | 16 |
| Bowel obstruction/process | 20 |
| Infection (meningitis, sepsis) | 25 |
| Bacterial process (atypicals, TB, PCP, Anthrax) | 30 |
| Missed fracture/disclocation | 35 |
| AAA | 40 |
| Diabetes (hypoglycemic) | 45 |
| Pregnant (ectopic) | 50 |
| Toxic Ingestion or reaction to medication | 55 |
| Intracranial process (mass, bleed, trauma) | 60 |
| Temporal arteritis | 65 |
| Testicular Torsion | 70 |
| CVA/stroke | 75 |
| Dehydration/electolyte abnormality | 77 |
| Vascular injury | 80 |
| Tendon injury | 85 |
| Retailed Foreign Body | 90 |
| Underlying infection or predispostion to infection | 95 |
| Unrecognized trauma | 100 |
| Airway obstruction (epiglottitis, FB) | 105 |
| Bronchaspasm/hypoxia | 110 |
| Bleeding abnormality | 115 |
| Anemia/hemorrhage | 120 |
| Spinal process (compression/disc) | 125 |
| Endocrinie (thyroid) | 130 |
| Glaucoma | 135 |
| PID | 140 |
| Suicidality assessed | 145 |
| Carbon monoxide | 150 |
| Seizure | 155 |
| Dysrythymia | 160 |
| Rhabdomyolosis | 165 |
| Cancer | 170 |

What is claimed is:

1. A method for generating one or more fail-safes in response to patient data; comprising:
storing a plurality of fail-safes, each fail-safe corresponding to potential high-risk diagnoses;
inputting at least one patient complaint;
inputting at least one physician diagnosis of the patient; and
generating a list of one or more patient fail-safes;
wherein the patient fail-safes correlate to either of the inputted patient complaint or the inputted physician diagnosis.

2. A method as recited in claim 1, further comprising:
ranking the patient fail-safes based on the severity of the potential high-risk diagnoses associated with each fail-safe.

3. A method as recited in claim 2, wherein the patient fail-safes are numerically weighted according to the severity of the potential high-risk diagnoses associated with each fail-safe.

4. A method as recited in claim 1, wherein the list of patient fail-safes contains fail-safes correlating to both the inputted patient complaint and the inputted physician diagnosis.

5. A method as recited in claim 2, wherein the patient fail-safes are generated as a checklist for review by a physician.

6. A method as recited in claim 5, wherein the highest ranking fail-safes are placed highest on the checklist.

7. A method as recited in claim 1, wherein inputting at least one patient complaint comprises selecting from a stored list of possible patient complaints.

8. A method as recited in claim 1, wherein inputting at least one physician diagnosis comprises selecting from a stored list of possible physician diagnoses.

9. A method for generating one or more fail-safes in response to patient data; comprising:
storing a plurality of fail-safes, each fail-safe correlating to potential high-risk diagnoses;
inputting at least one patient complaint;
generating a list of one or more patient fail-safes correlating to the inputted patient complaint; and
displaying the patient fail-safes in order based on the severity of the potential high-risk diagnoses associated with each fail-safe.

10. A method as recited in claim 9, further comprising:
inputting a physician diagnosis of the patient; and
generating a list of one or more patient fail-safes correlating to the inputted physician diagnosis.

11. A method as recited in claim 10, wherein the list of patient fail-safes contains fail safes correlating to both the inputted patient complaint and the inputted physician diagnosis.

12. A method as recited in claim 9, wherein the patient fail-safes are numerically weighted according to the severity of the potential high-risk diagnoses associated with each fail-safe.

13. A method as recited in claim 9, wherein the patient fail-safes are generated as a checklist for review by a physician.

14. A method as recited in claim 13, wherein the highest ranking fail-safes are placed highest on the checklist.

15. A method as recited in claim 9, wherein the patient fail-safes are output in an order based on the severity of the fail-safe and the frequency of the fail-safe occurrence.

16. A computer user interface for entering medical data of a patient, comprising:
a complaint field for entering a complaint expressed by the patient;
a diagnosis field for entering a potential diagnosis from the physician; and
a pane for displaying one or more fail-safes corresponding to potential high-risk diagnoses for consideration by the physician.

17. A computer user interface as recited in claim 16, wherein the one or more fail-safes are generated from the complaint field.

18. A computer user interface as recited in claim 16, wherein the one or more fail-safes are generated from the diagnosis field.

19. A computer user interface as recited in claim 16, wherein the one or more fail-safes are generated from the complaint field and the diagnosis field.

20. A computer user interface as recited in claim 16, wherein the complaint field is configured to be populated from a pre-selected list of patient complaints.

21. A computer user interface as recited in claim 16, wherein the diagnosis field is configured to be populated from a pre-selected list of potential diagnoses.

22. A computer user interface as recited in claim 16, wherein the displayed fail-safes are generated from a list of fail-safes correlating to both the inputted patient complaint and the inputted physician diagnosis.

23. A computer user interface as recited in claim 16, wherein the fail-safes are displayed according to the severity of the potential high-risk diagnoses associated with each fail-safe.

24. A computer user interface as recited in claim 23, wherein each fail-safe has a checkbox for recording that the physician considered the fail-safe in treating the patient.

25. A computer user interface as recited in claim 16, further comprising:
a patient data field for entering identification data of a patient.

26. A computer user interface as recited in claim 16, further comprising:
patient discharge field for displaying discharge instructions for the patient.

27. A medical diagnostic system comprising:
a user interface for entering and displaying patient data;
a database comprising a list of patient complaints;
a database comprising a list of potential physician diagnoses; and
a database comprising a list of fail-safes;
wherein said fail-safes correspond to potential high-risk diagnoses;
wherein each fail-safe correlates to at least one complaint or potential diagnosis; and
wherein the user interface is configured to display a list of fail-safes upon entry of a patient complaint or potential diagnosis.

28. A medical diagnostic system as recited in claim 27:
wherein the list of fail-safes are weighted according to severity of the potential high-risk diagnosis associated with each fail-safe; and
wherein the fail-safes are displayed in order of ranking with the highest-weighted fail-safes listed first.

29. A medical diagnostic system as recited in claim 27, wherein the user interface comprises a plurality of fields for entering at least one patient complaint and at least one potential physician diagnosis.

30. A medical diagnostic system as recited in claim 28, wherein the plurality of fields are configured to populate from either the list of patient complaints or the list of potential physician diagnoses.

31. A medical diagnostic system as recited in claim 27, further comprising a database comprising a list of discharge instructions.

32. A medical diagnostic system as recited in claim 27, further comprising a database comprising a list of physicians.

33. A medical diagnostic system as recited in claim 27, further comprising a plurality of terminals for displaying the user interface.

34. A medical diagnostic system as recited in claim 33, further comprising a site server configured to store said fail-safe database, patient complaint database, and potential physician diagnoses database.

35. A medical diagnostic system as recited in claim 34, further comprising:
a master server coupled to said site server via the Internet;
wherein said master server is capable of updating said site server.

36. An apparatus for generating one or more fail-safes in response to patient data; comprising:
a computer;
a database associated with said computer, said database storing a plurality of fail-safes, each fail-safe corresponding to potential high-risk diagnoses;
means for receiving input of at least one patient complaint;
means for receiving input of at least one physician diagnosis of the patient; and
means for generating a list of one or more patient fail-safes;
wherein the patient fail-safes correlate to either of the inputted patient complaint or the inputted physician diagnosis.

37. An apparatus as recited in claim 36, further comprising:
means for ranking the patient fail-safes based on the severity of the potential high-risk diagnoses associated with each fail-safe.

38. An apparatus as recited in claim 37, wherein the patient fail-safes are numerically weighted according to the severity of the potential high-risk diagnoses associated with each fail-safe.

39. An apparatus as recited in claim 36, wherein the fail-safes stored in the database correlate to both the inputted patient complaint and the inputted physician diagnosis.

40. An apparatus as recited in claim 36, wherein the least one patient complaint is selecting from a database of possible patient complaints.

41. An apparatus as recited in claim 36, wherein inputting at least one physician diagnosis comprises selecting from a stored list of possible physician diagnoses.
